SEQUENCE LISTING (16 sequences)

SEQ ID NO: 1 - DNA, 26 bp, Homo sapiens
tgtagttttcggagttagtgtcgcgc

SEQ ID NO: 2 - DNA, 30 bp, Homo sapiens
gttttgtagtttttggagttagtgttgtgt

SEQ ID NO: 3 - DNA, 25 bp, Homo sapiens
gggtcggagtttttcggagttgcgc

SEQ ID NO: 4 - DNA, 29 bp, Homo sapiens
ttttgggttggagttttttggagttgtgt

SEQ ID NO: 5 - DNA, 27 bp, Homo sapiens
gggtgatgttatcgtttttgtatcgac

SEQ ID NO: 6 - DNA, 29 bp, Homo sapiens
gggggtgatgttattgtttttgtattgat

SEQ ID NO: 7 - DNA, 26 bp, Homo sapiens
aagatttggcgttgggcgggacgttc

SEQ ID NO: 8 - DNA, 28 bp, Homo sapiens
gtaagatttggtgttgggtgggatgttt

SEQ ID NO: 9 - DNA, 25 bp, Homo sapiens
cctacgatcgaaaacgacgcgaacg

SEQ ID NO: 10 - DNA, 30 bp, Homo sapiens
ctcaacctacaatcaaaaacaacacaaaca

SEQ ID NO: 11 - DNA, 26 bp, Homo sapiens
ccgctctcttcgctaaatacgactcg

SEQ ID NO: 12 - DNA, 29 bp, Homo sapiens
aacccactctcttcactaaatacaactca

SEQ ID NO: 13 - DNA, 25 bp, Homo sapiens
cctcccctaacgtaaactcgaaacg

SEQ ID NO: 14 - DNA, 27 bp, Homo sapiens
cacctcccctaacataaactcaaaaca

SEQ ID NO: 15 - DNA, 25 bp, Homo sapiens
actccaacccgaacctcgccgtacg

SEQ ID NO: 16 - DNA, 28 bp, Homo sapiens
aaaactccaacccaaacctcaccataca

(19) United States
(12) Patent Application Publication
(10) Pub. No.: US 2009/0155786 A1
(43) Pub. Date: Jun. 18, 2009

(54) COMPOSITIONS AND METHODS FOR DETECTING MARKERS OF CANCER

(75) Inventors: Carmen J. Marsit, Cambridge, MA (US); Margaret R. Karagas, Cambridge, MA (US); Angeline S. Andrew, Cambridge, MA (US); Mei Liu, Cambridge, MA (US); Karl T. Kelsey, Cambridge, MA (US)

Correspondence Address: BURNS & LEVINSON, LLP, 125 SUMMER STREET, BOSTON, MA, 02110, US

(73) Assignee: PRESIDENT AND FELLOWS OF HARVARD COLLEGE, Cambridge, MA (US)

(21) Appl. No.: 12/063,942

(22) PCT Filed: Aug. 15, 2006
(86) PCT No.: PCT/US2006/031761

§ 371 Date: Oct. 23, 2008

Related U.S. Application Data
(60) Provisional application No. 60/708,823, filed on Aug. 15, 2005.

(51) Int. Cl. C12Q 1/68

(52) U.S. Cl. 435/6

(57) ABSTRACT

Disclosed herein are compositions and methods for detecting one or more markers indicative of cancer. In one instance the marker is one or more methylated genes, such as SFRPs. In another instance the marker is an altered protein, such as p53.

### RELATED APPLICATIONS

This application claims priority to and the benefit of U.S. Provisional Patent Application Ser. No. 60/708,823, filed Aug. 15, 2005.

### GOVERNMENT SUPPORT

This invention was partially supported by funds from the United States Government, specifically grants NIEHS ES00002, P42ES007373, P42ES005947, NCI grant R01 CA100679, and NIEHS toxicology and environmental health sciences training grant T32 ES007155, hence, the United States Government has certain rights to this invention.

### FIELD OF THE INVENTION

The present invention pertains to compositions and methods for detecting markers indicating the presence or predisposition of cancer.

### BACKGROUND OF THE INVENTION

In the United States in 2004, approximately 60,000 new cases of bladder cancer were diagnosed, and almost 13,000 deaths ascribed to this disease. The mortality of this disease is attributed to higher stage solid tumors which invade the muscular layers of the bladder. Clinically, biological markers that can predict this invasive phenotype would be useful for determining patient prognosis as well as in properly designing treatment regimens. Alteration of the TP53 tumor suppressor, as determined using protein immunohistochemistry, has also been associated with more aggressive, invasive bladder cancer, and has been suggested to clinically motivate more radical surgery or radiotherapy. However, the sensitivity and specificity of using p53 alterations alone in making clinical judgments has been questioned suggesting that additional biomarkers, which can be used alternatively or in conjunction with altered p53, are necessary. Regulation of cell migration is critical to maintaining a non-invasive phenotype. CTNNB1 (β-CATENIN), activated by the WNT pathway, acts as a transcription factor, as well as interacts with the cadherins and the cytoskeleton, mediating intercellular adhesion and playing an important role in cellular morphogenesis. Disruption of this pathway, through epigenetic inactivation of the Secreted Frizzled Receptor Proteins (SFRPs), secreted antagonists of WNT signaling, has been demonstrated in colorectal cancer and is responsible for the constitutive activation of WNT signaling in a number of primary colorectal tumors. In a hospital-based study of papillary bladder cancer, loss of SFRP1 expression is a marker of higher tumor stage and grade and poorer survival, with the loss of expression attributable to promoter hypermethylation and allelic loss of SFRP1. As appropriate function of the WNT and TP53 pathways may play an important biological role in maintaining a non-invasive phenotype, aberrant activation of these pathways through epigenetic silencing the SFRPs or alterations of TP53 expression, may be important in the determination of this invasive phenotype.

There is a clear need to develop methods for detecting markers for the presence or predisposition of cancer including, but not limited to, bladder cancer. This invention addresses this need.

### SUMMARY OF THE INVENTION

The present invention pertains to compositions and methods for detecting markers that indicate the presence or predisposition of cancer in an individual. These markers can be specific methylated genes as well as the presence of certain proteins in particular conformations (or configurations).

One embodiment of the present invention is directed toward the detection of one or more markers indicative of the presence or predisposition of cancer. In one aspect, the cancer is bladder cancer. In one aspect, the marker is one or more methylated SFRP genes. These genes encode for Secreted Frizzled Receptor Proteins (and hence are referred to as SFRP genes). In another aspect, the marker is one or more proteins including p53. In a particular aspect, the p53 protein is in an altered conformation.

Another embodiment is directed to kits that can be used for the detection of markers indicative of cancer. These markers include methylated genes and proteins in an altered conformation. In a particular aspect, the methylated gene is one or more SFRP genes. In another particular aspect, the protein is an altered p53. The kits comprise one or more reagents for ascertaining the methylation status of nucleic acids obtained from a biological sample. Optionally, these kits can comprise one or more reagents for amplifying the amount of nucleic acid, e.g., DNA present in the sample. Further, the kits can comprise reagents necessary to detect the presence of specific proteins in particular conformation. Such reagents are well known to those skilled in the art.

For a better understanding of the present invention, reference is made to the accompanying drawings, detailed description, and appended claims.

### BRIEF DESCRIPTION OF THE SEVERAL VIEWS OF THE DRAWING

FIG. 1 shows Kaplan-Meier survival probability curves in patients with bladder cancer by SFRP gene methylation status (1 or more SFRP genes methylated vs. none, N=351). Survival time is defined as the time from initial diagnosis to the patient's death. Tick marks represent censored values. The difference between the groups was tested by use of the log-rank method, and there was a significant difference in survival between those with any SFRP gene methylation compared to those with none (P<0.0003); and

FIG. 2 illustrates suitable SFRP primers for methylation-specific PCR.

### DETAILED DESCRIPTION OF THE INVENTION

The present invention pertains to compositions and methods for detecting markers that indicate the presence or predisposition of cancer in an individual. These markers can be specific methylated genes as well as certain proteins in particular conformations (or configurations).

To facilitate the understanding of the present invention, a number of terms and phrases are defined below:

As used herein, the terms “polynucleotide” and “oligonucleotide” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form.

A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be inputted into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “gene product” includes an amino acid, e.g., peptide or polypeptide, generated when a gene is transcribed and then translated.

A “primer” includes a short polynucleotide, generally with a free 3′-OH group that binds to a target or “template” present in a sample of interest by hybridizing with the target, and thereafter promoting polymerization of a polynucleotide complementary to the target.

A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, typically a thermally-stable polymerase enzyme. Methods for PCR are well known in the art, and are taught, for example, in MacPherson et al., IRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, for example, Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “polypeptide” includes a compound of two or more subunit amino acids, amino acid analogs, or peptidomimetics. The subunits may be linked by peptide bonds. In another embodiment, the subunit may be linked by other bonds, e.g., ester, ether, etc. As used herein the term “amino acid” includes either natural and/or unnatural or synthetic amino acids, including glycine and both the D or L optical isomers, and amino acid analogs and peptidomimetics. A peptide of three or more amino acids is commonly referred to as an oligopeptide. Peptide chains of greater than three or more amino acids are referred to as a polypeptide or a protein.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” The stringency of a hybridization reaction includes the difficulty with which any two nucleic acid molecules will hybridize to one another. Under stringent conditions, nucleic acid molecules at least 60%, 65%, 70%, 75% identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

As used herein, the term “nucleic acid molecule” is intended to include DNA molecules, e.g., cDNA or genomic DNA, and RNA molecules, e.g., mRNA, and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules, which are separated from other nucleic acid molecules that are present in the natural source of the nucleic acid. For example, with regards to genomic DNA, the term “isolated” includes nucleic acid molecules that are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

As used herein, the term configuration indicates the spatial arrangement in which atoms are covalently linked in a molecule; conformation is the three dimensional arrangement adopted by a molecule, usually a complex macromolecule. (Molecules with the same configuration can have more than one conformation.)

In the United States each year, almost 13,000 deaths are attributable to bladder cancer, with the majority of these deaths related to higher stage, muscle-invasive solid tumors. Epigenetic silencing of the Secreted Frizzled Receptor Proteins (SFRPs), antagonists of the WNT pathway, leads to constitutive WNT signaling, altering cell morphology and motility. Identifying alterations in this pathway in bladder cancer can prove useful for defining the invasive phenotype and provide targets for guiding therapy.

Secreted frizzled related proteins (SFRPs) are related to the frizzled family of transmembrane receptors. The SFRPs are approximately 30 kDa in size, and each contains a putative signal sequence, a frizzled-like cysteine-rich domain, and a conserved hydrophilic carboxy-terminal domain. It has been reported that SFRPs may function to modulate WNT signaling, or function as ligands for certain receptors. Rattner, et al., PNAS USA, 94(7):2859 2863 (1997), the entire teaching of which is incorporated herein by reference.

Frizzled (FZD) genes encode receptors for WNTs, which play key roles in carcinogenesis and embryogenesis. Homologues of the N-terminal region of frizzled exist either as transmembrane or secreted molecules. The Secreted Frizzled Related Protein 2 (SFRP2) is upregulated within 2 days of in vitro development. In vivo SFRP2 expression was likewise found in mesenchymal condensates and subsequent epithelial structures. Detailed in situ hybridization analysis revealed SFRP2 expression during development of the eye, brain, neural tube, craniofacial mesenchyme, joints, testis, pancreas and below the epithelia of oesophagus, aorta and ureter where smooth muscles develop. In a comparative analysis, transcripts of the related SFRP1 and SFRP4 genes were frequently found in the same tissues as SFRP2 with their expression domains overlapping in some instances, but mutually exclusive in others. While SFRP1 is specifically expressed in the embryonic metanephros, eye, brain, teeth, salivary gland and small intestine, there is only weak expression of SFRP4 except for the developing teeth, eye and salivary gland. Nevertheless, SFRP genes play quite distinct roles in the morphogenesis of several organ systems.

The WNTs are a family of secreted glycoproteins characterized by several conserved cysteine residues. See, Kawano, Y et al., J. Cell Science, 116(13), pp. 2627-2634, the entire teaching of which is incorporated herein by reference including the references cited therein. There are 19 human WNT genes, several of which encode additional, alternatively spliced isoforms. See, Miller, J. R. (2001) Genome Biol. 3, reviews 3001.1-3001.15, the entire teaching of which is incorporated herein by reference. WNT proteins have been grouped into two classes—canonical and non-canonical—on the basis of their activity in cell lines or in vivo assays. Canonical WNTS stabilize β-catenin, thereby activating transcription of Tcf/LEF target genes. This results in secondary axis formation in Xenopus embryos and morphological transformation of some mammalian cell lines. Noncanonical WNTs activate other signaling pathways, such as the planar-cell-polarity (PCP)-like pathway that guides cell movements during gastrulation and the WNT/Ca⁺² pathway. Noncanonical WNTs can even antagonize the canonical pathway. Several WNT proteins appear to have both canonical and noncanonical properties.

The WNT receptor complex that activates the canonical pathway contains two components: a member of the frizzled (Fz) family (there are 10 of these seven-transmembrane-span proteins in human) and either one of two single-span transmembrane proteins. (See Kawano, Y. et al.) Activation of the noncanonical WNT pathways is mediated by the Fz family WNT receptors.

WNT antagonists can be divided into two functional classes, the SFRP class and the Dickkopf class: members of the SFRP class, which includes the SFRP family, bind directly to WNTs, thereby altering their ability to bind to the WNT receptor complex. (See, Kawano, Y. et al.)

SFRPs are antagonists that directly bind to WNTs. It remains to be elucidated how SRFPs antagonize WNT signaling. There are presently eight known members of the family. (See, Kawano, Y. et al.) A unifying nomenclature now exists for five of these (SFRP1 to SFRP5). Members of the SFRP family have been cloned. (See Rattner, A. et al. (1997) PNAS 94, 2859-2863, the entire teaching of which is incorporated herein by reference.

In addition to its role during development, the WNT pathway plays an important role in proliferation, differentiation and apoptosis in adult tissues. (See, Kawano, Y. et al.) Thus aberrant activation of the WNT pathway has been found to occur during tumorigenesis. The frequent down-regulation of SFRPs in carcinomas and their up-regulation in some degenerative diseases points to their importance in controlling WNT activity in healthy tissue.

Recently, much interest has been focused on the biology of epigenetic phenomena, namely processes which alter the phenotype but which are not associated with changes in DNA sequence (Wolffe, Science 286:481-486 (1999), the entire teaching of which is incorporated herein by reference). One of the best characterized epigenetic processes is DNA methylation (Wolffe et al., Curr Biol. 10:R463-R465 (1999), the entire teaching of which is incorporated herein by reference).

Investigators have detected aberrantly methylated DNA from cancer patients. This has been reported for patients with a variety of cancers, including lung (Esteller, et al., Cancer Res 59(1):67 (1999), the entire teaching of which is incorporated herein by reference) and liver cancer (Wong et al., Cancer Res 59(1):71 (1999), the entire teaching of which is incorporated herein by reference).

The SFRP1 gene is found at chromosome 8p21, a site of frequent loss of heterozygosity in human tumors, Wright, K. et al. (1998) Oncogene 17, pp. 1185-1188, the entire teaching of which is incorporated herein by reference. Hypermethylation of the SFRP1 promoter (as well as other SFRPs) occurs at a high frequency in primary colorectal carcinomas, Suzuki, H. et al. (2002) Nat. Genet. 31, pp. 141-149, and Suzuki, H. et al. (2004) Nat. Genet. 36(4), pp. 417-422, the entire teachings of which are incorporated herein by reference—indicating the involvement of methylation in certain disease processes.

It is clear that inactivation of the p53 pathway is important in cancers such as bladder cancer, as the number of tumors having p53 mutations increases with the degree of invasiveness of the tumor. See, Kelsey, K. T., et al. Br J Cancer (2004) 90:1572-6, the entire teaching of which is incorporated herein by reference including the references cited therein. (As used herein “TP53” refers to the gene that encodes the p53 protein.) At the same time, a large fraction of early stage disease displays altered p53 staining, suggesting that the p53 pathway is disrupted in some fashion. The significance of this discordance is unclear; stabilization of nuclear protein could indicate that the protein is mutant, but might also reflect an altered activation pathway for p53 or disrupted turnover kinetics. P53 protein in the cell is tightly regulated, with negligible antibody staining noted in normal tissues. However, inhibition of degradation, mutation or enhanced production of p53 protein (or a combination thereof) could all result in antibody recognition of the protein and thus abnormal staining. Studies indicate that the presence of mutation at p53 markedly increases with stage and grade of disease, Spruk III C. H., et al. (1994) Cancer Res 54:784-788; Berggren P, et al. (2001) Br J Cancer 84:1505-1511; and Smith N. D., et al. (2003) J Urol 169:1219-1228, the entire teachings of which are incorporated herein by reference. Other studies that compared both immunohistochemical staining and p53 mutations found appreciable staining in tumors harboring mutations, see, e.g., Esrig D, et al., (1993) Am J Pathol 143:1389-1397, the entire teaching of which is incorporated herein by reference.

Using a population-based study of bladder cancer (N=355), investigators examined epigenetic alterations, specifically gene promoter hypermethylation, of four SFRP genes in addition to immunohistochemical staining of p53, which has been previously been demonstrated as a predictor of invasive disease. It was observed that a significant linear trend (P<0.0004) was established for the magnitude of the risk of invasive disease together with the number of SFRP genes methylated. Both p53 alteration and SFRP gene methylation showed significant independent associations with invasive bladder cancer. Strikingly, in examining the joint effect of these alterations, investigators observed a greater than 30-fold risk of invasive disease for patients with both altered SFRP gene methylation and intense p53 staining (Odds Ratio 32.1, P<10⁻¹³). Overall patient survival was significantly poorer in patients with any SFRP genes methylated (P<0.0003) and in proportional hazards modeling, patients with methylation of any SFRP gene had significantly poorer overall survival (HR 1.78, P<0.02) controlled for p53 staining intensity and other survival associated factors. Classifying tumors based on SFRP methylation status and p53 protein staining intensity may be a clinically powerful predictor of invasive, deadly disease.

One embodiment of the present invention is directed toward the detection of one or more markers indicative of cancer. In one aspect, the cancer is bladder cancer. Suitable markers include both nucleic acids and proteins. In one aspect of the present embodiment, the marker is a methylated gene. In a particular aspect, the gene(s) encodes Secreted Frizzled Receptor Proteins and hence is referred to as a SFRP gene(s). The particular markers can be selected from the group SFRP1 through SFRP5 as well as combinations thereof. In another aspect, the marker is a protein. In a particular aspect, the protein is p53. In a more particular aspect, the protein is p53 with an altered conformation.

In one aspect of the present embodiment, a method for detecting DNA methylation in a gene is described. The method comprises obtaining a biological sample from an individual which has nucleic acid molecules (including DNA). This sample can be, for example, tissues or cells from any organ system. In a particular aspect, the tissue or cells are obtained from the tissue of interest, e.g., the bladder. In a further aspect, the biological sample is obtained from the muscle layer of the bladder. Once the sample has been obtained, isolation of nucleic acids can be accomplished using methods well known to those skilled in the art. The nucleic acid can be examined for methylation. In one aspect, SFRP genes can be examined for methylation.

In another aspect of the present embodiment, detection of methylated genes, such as SFRP, can be combined with the detection of p53 protein. Detection of p53 protein in a biological sample can be accomplished using well known immunohistochemistry. The presence of p53 has been linked to cancer (see above). One or more biological samples can be obtained from an individual and screened for both methylated genes, such as SFRP, and the presence of altered p53 protein. The Example section provides for one protocol to effectuate this aspect of the invention.

### DNA Extraction:

Nucleic acid, such as DNA, can be extracted from suitable tissue employing methods well known to those skilled in the art. For example, one or more 20 μM sections can be cut from each fixed, paraffin-embedded sample putatively containing tumor and transferred into microcentrifuge tubes. The paraffin can then be dissolved using Histochoice Clearing Agent (Sigma-Aldrich, St. Louis, Mo.) followed by 2 washes with 100% ethanol and 1 wash with PBS. The samples can then be incubated in SDS-lysis solution (approximately, 50 mM Tris-HCl, pH 8.1, 10 mM EDTA, 1% SDS) with proteinase K (Qiagen, Valencia, Calif.) overnight at approximately 55° C. De-crosslinked can next be accomplished using NaCl (final concentration of about 0.7 M) and incubating at around 65° C. for approximately 4 hours. DNA can be recovered using, e.g., the Wizard DNA clean-up kit (Promega, Madison, W) according to the manufacturer's protocols—or any suitable clean-up kit.

Sodium bisulfite modification of the DNA can be accomplished using, e.g., the EZ DNA Methylation Kit (Zymo Research, Orange, Calif.) following the manufacturer's protocol, with the addition of around 5 minute initial incubation at about 95° C. prior to the addition of the denaturation reagent. The de-crosslinking incubation as well as the 95° C. incubation ensure more complete melting of the DNA and thus more complete sodium bisulfite conversion.

### Methylation-Specific PCR:

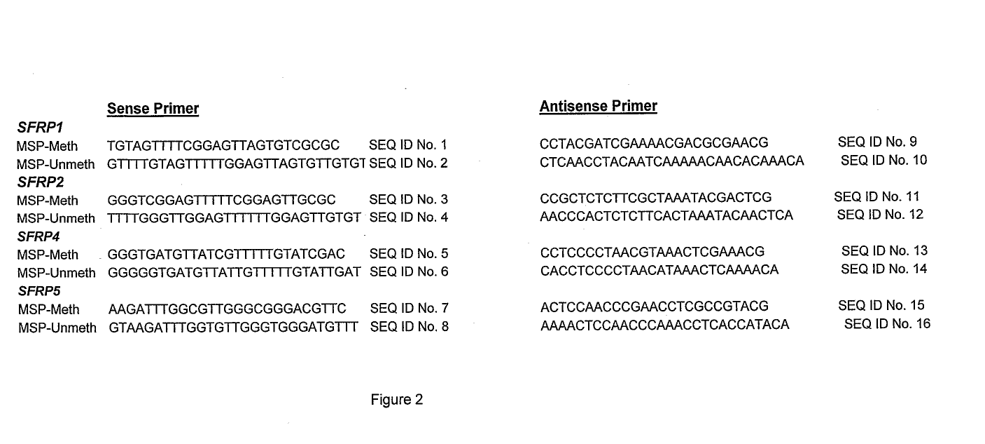

The sodium bisulfite modified DNA can be used as the template for methylation-specific PCR (MS-PCR) as previously described (Herman J G, et al. (1996) PNAS USA 93:9821-6, the entire teaching of which is incorporated herein by reference) using primers specific for the methylated promoter of SFRP1, SFRP2, SFRP4, and SFRP5 (see, Suzuki H, et al. (2004) Nat Genet 36:417-22, the entire teaching of which is incorporated herein by reference). Examples of suitable primers are illustrated in FIG. 2. All methylation-specific PCRs can be optimized to detect greater than 5% methylated substrate in each sample. To control for the presence of modified DNA, primers specific to a modified region of the ACTB genes containing no CpG sites can be used, Eads CA, et al. (2000) Nucleic Acids Res 28:E32, the entire teaching of which is incorporated herein by reference. Modified circulating blood lymphocyte DNA and that same lymphocyte DNA completely methylated using SssI DNA methylase and modified by treatment with sodium bisulfite can be used as the negative and positive controls, respectively, in each run.

### Quantitative Methylation Specific PCR:

Sodium bisulfite modified DNA from a number of invasive stage and a number of non-invasive stage tumors (randomly selected from among each category and analyzed blindly) can be subjected to quantitative RT-PCR using primers and, e.g., a Taqman Probe (Applied Biosystems, Foster City, Calif.) specific to modified ACTB as previously described, Eads CA, et al. (2000) Nucleic Acids Res 28:E32. Bisulfite modified human sperm DNA can serve as a positive control and a no template control can also run with the samples.

### Immunohistochemistry:

The immunohistochemical detection of p53 has been previously described, Kelsey K T, et al. (2004) Br J Cancer 90:1572-6, the entire teaching of which is incorporated herein by reference. Immunohistochemical staining of paraffin-embedded slides prepared from suitable tissue can be performed using the avidin-biotin complex technique well know to those skilled in the art. Slides can be deparaffinized and hydrated into water. Slides can undergo antigen retrieval in, e.g., Citra solution using, e.g., the Biocare Declocking Chamber (Biocare Medical, Walnut Creek, Calif.). Staining of protein, e.g., p53 can be performed using an antibody specific for that protein. For example, a monoclonal antibody from BioGenex (San Ramon, Calif.) can be used for staining p53 (for purposes of this invention, the mAb is referred to as “mAbP53” (e.g., a commercial antibody is referred to as p53 DO-1 mouse monoclonal antibody, available from Abcam, Novus, etc.). Staining of p53 can be performed using, e.g., a 1:100 dilution on, e.g., an Optimax I-6000 Immunostainer (BioGenex). The intensity of nuclear staining can be graded on a semiquantitative scale, e.g., from 0-3, rating intensity in the dominant pattern within the specimen.

### Statistical Analysis:

A multivariate unconditional logistic regression with methylation of any SFRP gene (1 or more versus 0) as the dependent variable to examine associations between patient demographics, exposure history, and tumor characteristics with SFRP gene methylation while controlling for possible confounding can be employed. The effects of multiple predictors on tumor invasion (i.e., invasive versus non-invasive) can be examined using unconditional logistic regression analysis, with adjustment for potential confounders; in the analysis, the relative risks of invasive disease can be estimated for having 1, 2, or 3 to 4 SFRP genes methylated as well as p53 staining intensity. To examine the joint effect of p53 alteration and SFRP gene methylation on tumor invasiveness, an analysis stratified by both SFRP gene methylation and p53 status with p53 negative (around <3 staining intensity) and no SFRP genes methylated as the reference category, again using unconditional logistic regression with adjustment for multiple covariates (i.e., age and sex) can be conducted.

Subject survival can be examined using Kaplan-Meier survival probability curves, and differences between strata tested using the log-rank test. In order to control for additional variables related to patient survival, Cox's proportional hazards modeling can be employed, using the same variables utilized in the logistic regression analysis. All P values preferably represent two-sided statistical tests.

A nucleic acid molecule of the present invention, e.g., a nucleic acid molecule having the nucleotide sequence of SEQ ID NO. 1-6, or a portion thereof, can be isolated using standard molecular biology techniques and the sequence information provided herein. Using all or portion of the nucleic acid sequence of SEQ ID NO. 1-16 as a hybridization probe, a molecule comprising SEQ ID NO. 1-16 can be isolated using standard hybridization and cloning techniques as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989. Further, nucleic acids of the present invention, e.g., SEQ ID NO. 1-16, can be employed as primers for PCR.

A nucleic acid of the invention can be amplified using cDNA, mRNA or alternatively, genomic DNA, as a template and appropriate oligonucleotide primers, such as SEQ ID NO. 1-16, according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g., using an automated DNA synthesizer.

In another embodiment, an isolated nucleic acid molecule of the invention comprises a nucleic acid molecule which is a complement of the nucleotide sequence of SEQ ID NO. 1-16, or a portion thereof. A nucleic acid molecule that is complementary to such a nucleotide sequence is one which is sufficiently complementary to the nucleotide sequence such that it can hybridize to the nucleotide sequence, thereby forming a stable duplex.

The nucleic acid molecule of the invention, moreover, can comprise only a portion of the nucleic acid sequence of SEQ ID NO 1-16 of the invention, or a fragment which can be used as a probe or primer. The probe/primer typically comprises substantially purified oligonucleotide.

Probes based on the nucleotide sequence of a nucleic acid molecule encoding SEQ ID NO 1-16 can comprise a labeling group attached thereto, e.g., the labeling group can be a radioisotope, a fluorescent compound, an enzyme, or an enzyme co-factor. Such probes can be used as a part of a diagnostic test kit for identifying cells or tissue which misexpresses, e.g., over- or under-express, a polypeptide of the invention, or which have greater or fewer copies of a gene of the invention.

As used herein, the term “hybridizes under stringent conditions” is intended to describe conditions for hybridization and washing under which nucleotide sequences at least 60% homologous to each other typically remain hybridized to each other. Preferably, the conditions are such that sequences at least about 70%, more preferably at least about 80%, even more preferably at least about 85% or 90% homologous to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60° C., and even more preferably at 65° C. Preferably, an isolated nucleic acid molecule of the invention that hybridizes under stringent conditions to the sequence of SEQ ID NO. 1-16. As used herein, a “naturally-occurring” nucleic acid molecule includes an RNA or DNA molecule having a nucleotide sequence that occurs in nature, e.g., encodes a natural protein.

In other embodiments, the oligonucleotides of the invention can include other appended groups such as peptides, e.g., for targeting host cell receptors in vivo, or agents facilitating transport across the cell membrane (see, e.g., Letsinger et al. (1989) Proc. Natl. Acad. Sci. USA 86:6553-6556; Lemaitre et al. (1987) Proc. Natl. Acad. Sci. USA 84:648-652; PCT Publication No. W088/09810) or the blood-brain barrier (see, e.g., PCT Publication No. W0 89/10134). In addition, oligonucleotides can be modified with hybridization-triggered cleavage agents (see, Krol et al. (1988) Bio-Techniques 6:958-976) or intercalating agents (see, Zon (1988) Pharm. Res. 5:539-549). To this end, the oligonucleotide may be conjugated to another molecule, e.g., a peptide, hybridization triggered cross-linking agent, transport agent, or hybridization-triggered cleavage agent. Finally, the oligonucleotide may be detectably labeled, either such that the label is detected by the addition of another reagent, e.g., a substrate for an enzymatic label, or is detectable immediately upon hybridization of the nucleotide, e.g., a radioactive label or a fluorescent label, e.g., a molecular beacon as described in U.S. Pat. No. 5,876,930.

Another embodiment is directed to kits that can be used for the detection of markers indicative of cancer. These markers include methylated genes and proteins in an altered conformation. In a particular aspect, the methylated gene is one or more SFRP genes. In another particular aspect, the protein is an altered p53. The kits comprise one or more reagents for ascertaining the methylation status of nucleic acids obtained from a biological sample. Optionally, these kits can comprise one or more reagents for amplifying the amount of nucleic acid, e.g., DNA present in the sample. Further, the kits can comprise reagents necessary to detect the presence of specific proteins in particular conformation. Such reagents are well known to those skilled in the art.

Of course, one skilled in the art will appreciate further features and advantages of the invention based on the above-described embodiments. Accordingly, the invention is not to be limited by what has been particularly shown and described, except as indicated by the appended claims.

### EXAMPLES

### Example 1

### The New Hampshire Study

### Study Population:

Residents of New Hampshire ages 25 to 74 years, diagnosed from Jul. 1, 1994 to Jun. 30, 1998 with cancer were identified by the rapid reporting system of the New Hampshire State Cancer Registry, as by state law, practitioners are required to provide a report of an incident cancer upon its diagnosis, Karagas M R, et al. (998) Environ Health Perspect 106 Suppl 4:1047-50, the entire teaching of which is incorporated herein by reference. All study participants were consented under the appropriate institutional review board protocols. Study participants underwent a personal interview to obtain information on demographic traits, use of tobacco (including frequency, duration, and intensity of cigarette smoking), and use of hair dyes. Pathology material was obtained from a total of 355 patients. Pathology reports and paraffin-embedded tumor specimens were requested from the treating physician/pathology laboratories, and tumor samples are obtained from the procedure involved in the initial diagnosis. Bladder tumors were reviewed by the study pathologist and classified according to the 1973 and 2004 WHO guidelines for bladder tumors. Additionally, the proportion of malignant cells in each sample was recorded for each sample, averaging 69% (median 80%). Patient survival was assessed using clinical records and the Social Security Death Index.

### DNA Extraction and Sodium Bisulfite Modification:

Three 20 μM sections were cut from each fixed, paraffin-embedded tumor sample and transferred into microcentrifuge tubes. The paraffin was dissolved using Histochoice Clearing Agent (Sigma-Aldrich, St. Louis, Mo.) followed by 2 washes with 100% ethanol and 1 wash with PBS. The samples were then incubated in SDS-lysis solution (50 mM Tris-HCl, pH 8.1, 10 mM EDTA, 1% SDS) with proteinase K (Qiagen, Valencia, Calif.) overnight at 55° C. De-crosslinked was performed by adding NaCl (final concentration 0.7 M) and incubating at 65° C. for 4 hours. DNA was recovered using the Wizard DNA clean-up kit (Promega, Madison, Wis.) according to the manufacturer's protocols. Sodium bisulfite modification of the DNA was performed using the EZ DNA Methylation Kit (Zymo Research, Orange, Calif.) following the manufacturer's protocol, with the addition of a 5 minute initial incubation at 95° C. prior to addition of the denaturation reagent. The de-crosslinking incubation as well as the 95° C. incubation ensure more complete melting of the DNA and thus more complete sodium bisulfite conversion.

### Methylation-Specific PCR:

Sodium bisulfite modified DNA was used as the template for methylation-specific PCR (MS-PCR) as previously described (Herman J G, et al. (1996) PNAS USA 93:9821-6) using primers specific for the methylated promoter of SFRP 1, SFRP2, SFRP4, and SFRP5. All methylation-specific PCRs are optimized to detect greater than 5% methylated substrate in each sample. To control for the presence of modified DNA, primers specific to a modified region of the ACTB genes containing no CpG sites were used. Modified circulating blood lymphocyte DNA and that same lymphocyte DNA completely methylated using SssI DNA methylase and modified by treatment with sodium bisulfite were used as the negative and positive controls, respectively, in each run.

### Quantitative Methylation Specific PCR:

Sodium bisulfite modified DNA from 10 invasive stage and 10 non-invasive stage tumors (randomly selected from among each category and analyzed blindly) was subjected to quantitative RT-PCR using primers and a Taqman Probe (Applied Biosystems, Foster City, Calif.) specific to modified ACTB as previously described. Bisulfite modified human sperm DNA served as a positive control and a no template control was also run with the samples. All samples and controls were run in duplicate.

### p53 Immunohistochemistry:

The immunohistochemical detection of p53 has been previously described, Kelsey K T, et al. (2004) Br J Cancer 90:1572-6.

### Statistical Analysis:

We used multivariate unconditional logistic regression with methylation of any SFRP gene (1 or more versus 0) as the dependent variable to examine associations between patient demographics, exposure history, and tumor characteristics with SFRP gene methylation while controlling for possible confounding. We examined the effects of multiple predictors on tumor invasion (i.e., invasive versus non-invasive) using unconditional logistic regression analysis, with adjustment for potential confounders; in the analysis, the relative risks of invasive disease were estimated for having 1, 2, or 3 to 4 SFRP genes methylated as well as p53 staining intensity. To examine the joint effect of p53 alteration and SFRP gene methylation on tumor invasiveness, we conducted an analysis stratified by both SFRP gene methylation and p53 status with p53 negative (<3 staining intensity) and no SFRP genes methylated as the reference category, again using unconditional logistic regression with adjustment for multiple covariates (i.e., age and sex).

Patient survival was examined using Kaplan-Meier survival probability curves, and differences between strata tested using the log-rank test. In order to control for additional variables related to patient survival, Cox's proportional hazards modeling was employed, using the same variables utilized in the logistic regression analysis. All P values represent two-sided statistical tests.

### Results & Discussion:

As expression of SFRP1, SFRP2, SFRP4, and SFRP5 has been previously demonstrated in normal bladder epithelium, Dyrskjot L, et al. (2004) Cancer Res 64:4040-8, the entire teaching of which is incorporated herein by reference, promoter DNA hypermethylation indicative of epigenetic silencing of these genes was determined, using methylation-specific PCR (MS-PCR), in a population-based case series study of bladder cancer in New Hampshire. The prevalence of methylation silencing were 18% (64/355), 52% (184/355), 9% (32/355), and 37% (132/355), respectively for SFRP1, SFRP2, SFRP4, and SFRP5. Methylation of any of the SFRP genes occurred in 62% (221/355) of the tumors examined. To assure that there were no biases in the detection of promoter methylation attributable to the proportion of tumorous tissue in the substrate used for MS-PCR, we examined methylation of any SFRP gene and the proportion of malignant tissue in individual samples, assessed by an experienced urologic pathologist. There was no relationship between detection of promoter methylation and the percent of tumorous tissue in the sample (P<0.8). This result is consistent with the previously reported finding (in the same samples) of no relationship between proportion of tumorous tissue and the measured presence of persistent p53 protein by IHC or TP53 mutation, Kelsey K T, et al. (2005) Int J Cancer, in press, the entire teaching of which is incorporated herein by reference. In order to ensure that there was no bias in the amplification of substrate by tumor stage, we also performed quantitative RT-PCR on a random subset of samples (10 invasive stage, 10 non-invasive stage) using the primers and probe for modified ACTB. The cycle threshold (Ct) for invasive tumors was 34.3 (SD 2.3) and for non-invasive tumors was 34.8 (SD 3.0). There was no significant difference (Student's T-test; p>0.6) in the substrate amplification by tumor stage, confirming the absence of bias in PCR amplifiable substrate.

Table 1 examines the association between having any SFRP gene methylated and demographics of the cases or tumor characteristics. Men had a significantly higher prevalence of any SFRP gene methylation compared to women, although there appeared to be no significant difference in the prevalence of any SFRP gene methylation by patient age or by histology, although this analysis is limited to the small number of non-transitional cell carcinomas available. Methylation of the SFRP genes was positively associated with advanced tumor stage (adjusted Odds Ratio [OR] for SFRP methylation among invasive tumors 2.8, 95% confidence interval [CI] 1.4-5.6). The prevalence of SFRP gene methylation was almost two-fold greater in current smokers compared to never or former smokers. When the metrics of smoking were examined in current smokers, there was no significant association between SFRP methylation and the intensity or duration of smoking.

TABLE 1
Bladder tumor SFRP gene methylation by patient
demographics and tumor characteristics.

| | Any SFRP Methylation | | | |
|---|---|---|---|---|
| | No Methyl- | Any methyl- | | |
| | ation | ation | | |
| Charac- | N = 134 | N = 221 | Adjusted OR* | Totals |
| teristic | N (%) | N (%) | (95% CI) | N = 355 |

Gender

| | | | | | | | |
|---|---|---|---|---|---|---|---|
| Female | 37 | (27.6) | 40 | (18.1) | 1.0 | (reference) | 77 |
| Male | 97 | (72.4) | 181 | (81.9) | 2.0 | (1.1-3.5) | 278 |

Age

| | | | | | | | |
|---|---|---|---|---|---|---|---|
| <65 | 68 | (50.7) | 106 | (48.0) | 1.0 | (reference) | 174 |
| ≧65 | 66 | (49.3) | 115 | (52.0) | 1.3 | (0.8-2.0) | 181 |

Histology^{†}

| | | | | | | | |
|---|---|---|---|---|---|---|---|
| Transi- | 130 | (97.7) | 210 | (96.3) | 1.0 | (reference) | 340 |
| tional | | | | | | | |
| Cell | | | | | | | |
| Carcinoma | | | | | | | |
| Non- | 3 | (2.3) | 8 | (3.7) | 0.7 | (0.07-8.1) | 11 |
| transi- | | | | | | | |
| tional | | | | | | | |
| Cell | | | | | | | |

Tumor Stage^{‡}

| | | | | | | | |
|---|---|---|---|---|---|---|---|
| Non- | 108 | (82.4) | 134 | (61.7) | 1.0 | (reference) | 242 |
| invasive | | | | | | | |
| Carcinoma | 6 | (4.6) | 6 | (2.8) | 0.5 | (0.1-1.9) | 12 |
| in-situ | | | | | | | |
| Invasive | 17 | (13.0) | 77 | (35.5) | 2.8 | (1.4-5.6) | 94 |

p53 Staining Intensity^{§}

| | | | | | | | |
|---|---|---|---|---|---|---|---|
| Score <3 | 102 | (82.3) | 143 | (69.4) | 1.0 | (reference) | 245 |
| Score 3+ | 22 | (17.7) | 63 | (30.6) | 1.3 | (0.6-2.6) | 85 |

Current Smoker

| | | | | | | | |
|---|---|---|---|---|---|---|---|
| No | 101 | (75.4) | 135 | (61.1) | 1.0 | (reference) | 236 |
| Yes | 33 | (24.6) | 86 | (38.9) | 1.9 | (1.1-3.3) | 119 |

*Odds Ratios (OR) adjusted for all other variables in the table and limited to subjects with complete data for all variable (N = 330).
^{†}4 tumors were missing histological classification
^{‡}7 tumors were missing stage classification
^{§}p53 immunohistochemistry was performed on 330 tumors.

We have previously reported that p53 immunohistochemical staining intensity is associated with invasive disease in these tumors, Kelsey K T, et al. (2004) Br J Cancer 90:1572-6. After controlling for potential confounders, p53 staining was not significantly associated with SFRP gene methylation, suggesting that epigenetic silencing of the SFRP genes occurs independent of alterations to p53.

As p53 alterations and SFRP gene methylation are unrelated, we examined their association with invasive bladder cancer. We used invasive disease as the dependent variable, and included variables for the number of SFRP gene methylated, p53 staining intensity (3+ vs. <3), age (<65 vs. ≧65 years), and gender in the multivariate logistic regression model. Current cigarette smoking was initially included in the model, due to its association with SFRP gene methylation. Tests for confounding showed that smoking was not significantly associated with invasiveness, and its removal from the model had no effect on the point estimates.

Table 2 shows a highly significant trend in the magnitude of the odds of having invasive bladder cancer with increasing numbers of SFRP genes silenced by methylation (P<0.0004).

TABLE 2
Methylation of multiple SFRP genes and altered p53 status
are independently associated with invasive bladder cancer.

| | | |
|---|---|---|
| | | Invasive Bladder Cancer* |
| Co-variate | N (N invasive disease) | N = 320 OR (95% CI)^{†} |

Number of SFRP Genes Methylated

| | | | | |
|---|---|---|---|---|
| 0 | 119 | (17) | 1.0 | (ref.) |
| 1 | 84 | (21) | 1.9 | (0.8-4.5) |
| 2 | 62 | (24) | 3.5 | (1.5-8.4) |
| 3 or 4 | 55 | (28) | 4.2 | (1.7-10.2)^{‡} |

p53 Alteration (Staining Intensity)

| | | | | |
|---|---|---|---|---|
| <3 | 243 | (33) | 1.0 | (ref.) |
| 3+ | 77 | (57) | 16.8 | (8.7-32.4) |

*Model predicting invasive disease compared to non-invasive. Carcinoma-in-situ were excluded from the model.
^{†}Model is controlled for age, sex, and all co-variates in the table.
^{‡}The trend for increasing magnitude in the relative risk of invasive disease with increasing number of SFRP genes methylated is significant (P < 0.0004).

This result indicates a very strong and significant relationship between methylation of these genes and invasive disease. Having one SFRP gene methylated imparts an almost 2-fold relative risk of invasive disease, while having 3-4 SFRP genes methylated imparts a 4.2-fold relative risk of invasiveness (controlled for potential confounders in the model). This model also shows a strong, significant, and independent association between p53 staining intensity and invasive disease. After adjusting for SFRP gene methylation as well as age and gender, patients with intense p53 staining were almost 17 times as likely to have invasive disease, compared to patients with less intensely staining tumors. The significant trend, showing that as the number of SFRP genes methylated increases the likelihood of the tumor to be invasive increases suggests this pathway is crucial for facilitating this invasive phenotype. That is, as more of these antagonists become silenced, the pathway can become more constitutively active and the clone more likely to become invasive. However, Salem, et al. (2000) Cancer Res 60:2473-6, the entire teaching of which is incorporated herein by reference, have previously noted that invasive bladder tumors have greater numbers of methylated CpG islands, suggesting these tumors may have a methylator phenotype. Interestingly, the methylation of the individual islands studied by Salem et al. occurred independently, while the methylation of SFRP loci is significantly correlated (data not shown); this is more suggestive of selection of silencing of the WNT pathway, rather than action of a less targeted, global epigenetic mechanism.

Next, we investigated whether SFRP gene methylation status may enhance the association between p53 staining intensity and invasive disease in a more than additive fashion. Table 3 shows the results of stratified multivariate logistic analyses, again using invasive bladder cancer as the dependent variable, and p53 staining intensity as the predictor. In tumors having no SFRP genes methylated, high p53 staining intensity imparted a 9.4-fold relative risk of invasive bladder cancer (95% CI 2.8-31.8), compared to low p53 staining intensity, controlled for age and gender. On the other hand, in tumors with one or more SFRP genes methylated, high p53 staining intensity had an OR for invasive bladder cancer of 32.1 (95% CI 12.9-79.8, P<10⁻¹³). This striking result suggests that tumors with SFRP methylation and TP53 pathway alteration have more than 30 times the odds of being invasive than tumors with no or low p53 staining intensity.

TABLE 3
Stratified Analysis of the Association between p53 Alteration
and Invasive Bladder Cancer by Methylation of any SFRP gene.

| | | | |
|---|---|---|---|
| | N | Invasive | |
| p53 Status | (N Invasive | Bladder Cancer | |
| (IHC Intensity) | Disease) | OR (95% CI) | P |

No SFRP Methylation

| | | | | | |
|---|---|---|---|---|---|
| p53 WT | 101 | (9) | 1.0 | (ref) | |
| p53 Altered | 18 | (8) | 9.4 | (2.8-31.8) | 0.0003 |

Positive SFRP Methylation

| | | | | | |
|---|---|---|---|---|---|
| p53 WT | 142 | (24) | 1.0 | (ref) | |
| p53 Altered | 59 | (49) | 32.1 | (12.9-79.8) | <10⁻¹³ |

Note:
Both models are controlled for age and sex. Carcinoma-in-situ were excluded from the models.

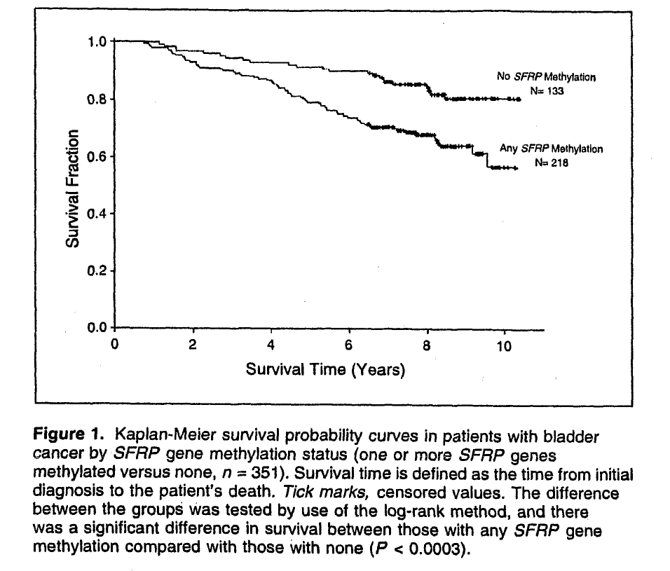

Finally, as silencing of the SFRP genes was associated with invasive disease, we examined the impact of these alterations on overall patient survival. FIG. 1 shows the Kaplan-Meier survival probability plots stratified by methylation of any SFRP gene. The log-rank test indicated a significant (P<0.0003) difference in survival between the strata. The covariate-adjusted hazard ratios and 95% CIs for the association between these variables and patient overall survival is give in Table 4. Patient age and carcinoma-in-situ histological stage showed significant associations with patient survival, while being male and having invasive stage disease also showed elevated instantaneous risks of death. Methylation of any SFRP gene imparted a 1.78 fold instantaneous risk of death (95% CI 1.08-2.92), yet TP53 staining intensity, in this model, has no significant association with patient survival.

TABLE 4
Methylation of any SFRP genes is associated
with reduced survival time.

| | | | |
|---|---|---|---|
| Variable | N | Hazard Ratio (95% CI) | P |

Age

| | | | | |
|---|---|---|---|---|
| <65 | 162 | 1.0 | (ref.) | |
| >65 | 168 | 2.43 | (1.54-3.82) | 0.0001 |

Gender

| | | | | |
|---|---|---|---|---|
| Female | 76 | 1.0 | (ref.) | |
| Male | 254 | 1.78 | (0.96-3.29) | 0.07 |

Tumor Stage

| | | | | |
|---|---|---|---|---|
| Non-invasive | 230 | 1.0 | (ref.) | |
| Carcinoma-in-situ | 10 | 3.86 | (1.62-9.20) | 0.002 |
| Invasive | 90 | 1.59 | (0.94-2.69) | 0.09 |

Any SFRP gene methylation

| | | | | |
|---|---|---|---|---|
| No | 124 | 1.0 | (ref.) | |
| Yes | 206 | 1.78 | (1.08-2.92) | 0.02 |

p53 Staining Intensity

| | | | | |
|---|---|---|---|---|
| <3 | 245 | 1.0 | (ref.) | |
| 3+ | 85 | 1.12 | (0.67-1.88) | 0.7 |

Note:
Model is controlled for all variables in table.

Although direct somatic mutations of APC or CYANB1, integral components of the WNT pathway, are uncommon in bladder cancer, Stoehr R, et al. (2002) Int J Oncol 20:905-11, the entire teaching of which is incorporated herein by reference, epigenetic silencing of pathway antagonists, the SFRPs occurs commonly. Our data shows that silencing of these genes occurs in approximately 60% of tumors and occurs more often in men. We and others have previously reported associations between smoking exposure and DNA methylation in lung cancer, Toyooka S, et al. (2003) Int J Cancer 103:153-60, and Kim DH, et al. (2001) Cancer Res 61:3419-24, the entire teachings of which are incorporated herein by reference. The current results, showing an association between SFRP gene methylation and smoking at the time of diagnosis, suggest that continuous exposure of the cancerous field to tobacco-smoke carcinogens is able to select for epigenetic silencing of these genes.

Importantly, alterations of p53 and epigenetic silencing of the SFRPs are both independently significantly related to the invasive phenotype of bladder cancer. As the majority of mortality in bladder cancer is associated with invasive disease, we observe a significant association between SFRP gene methylation and overall patient survival. It is of interest that p53 staining is not significantly associated with survival in the adjusted analysis, but may indicate its co-linearity with tumor stage. Our study indicates that classifying tumors based upon both SFRP gene methylation status and p53 immunohistochemistry would provide significantly improved clinical estimates of an aggressive and potentially fatal phenotype, better identifying patients in need of more aggressive treatment of their disease. Indeed, detection of tumor DNA methylation in serum is being proposed as a tool for cancer detection and follow-up, and detection of tumor-associated methylation in urine-derived DNA also holds promise for clinical application, Esteller M (2003) Lancet Oncol 4:351-8, and Friedrich M G, et al. (2004) Clin Cancer Res 10:7457-65, the entire teachings of which are incorporated herein by reference. Our results demonstrate that the SFRP genes are strong candidates for clinical use in these types of serum or urine diagnostics, as they may greatly aid in determining which patients will develop invasive disease.

### Example 2

### Utility of SFRP Gene Methylation as a Marker in Urine Sediment

The utility of the promoter hypermethylation of the 4 SFRP genes was also examined in matched samples of urine and bladder tumor tissue from patients undergoing surgical resection of bladder tumors. A total of 11 individual patients matched tumor/urine pairs were examined for promoter hypermethylation of SFRP1, SFRP2, SFRP4, and SFRP5. The urine was centrifuged at 2000 g for 15 min to pellet the sediment, and the supernatant discarded. DNA was isolated from the resulting sediment pellet using a Qiagen DNEasy Tissue DNA extraction kit (Qiagen, Valencia, Calif.) following the manufacturer's protocol. The resulting DNA was subjected to sodium bisulfite modification using the EZ DNA Methylation Kit (Zymo Research, Orange, Calif.) following the manufacturer's protocol. Tumor derived DNA was extracted and modified as has been previously described (see Marsit, et al. Cancer Res 2005, the entire teaching of which is incorporated herein by reference). Methylation-specific PCR to detect promoter hypermethylation of SFRP1, SFRP2, SFRP4, and SFRP5 was performed as described in Marsit et al. (Cancer Res 2005). Of the 44 possible comparisons (4 genes×11 tumor/urine pairs), 34 (77%) showed concordant patterns of methylation resulting in a specificity of 0.79 and a sensitivity of 0.73 for these tests.

What is claimed is:

1. A method of detecting one or more markers indicative of cancer in an individual, comprising:
(a) obtaining a suitable biological sample;
(b) isolating nucleic acid from said biological sample of (a);
(c) subjecting said isolated nucleic acid of (b) to methylation-specific PCR; and
(d) detecting said one or more markers in said individual.

2. The method of claim 1, wherein said one or more markers is a SFRP nucleotide sequence that is methylated.

3. The method of claim 2, wherein said SFRP nucleotide sequence is selected from the group consisting of SFRP1, SFRP2, SFRP4, SFRP5, and combinations thereof.

4. The method of claim 1, wherein said cancer is bladder cancer.

5. The method of claim 1, wherein said biological sample is muscle from a bladder of said individual.

6. The method of claim 1, wherein said isolated nucleic acid is DNA.

7. The method of claim 1, wherein said methylation-specific PCR is performed using one or more primers selected from the group consisting of SEQ ID NOs 1-16, and a combination thereof.

8. The method of claim 1, wherein step (b) further comprises isolating a suitable tissue sample from (a), and wherein step (c) further comprises subjecting said tissue sample to immunohistochemistry analysis.

9. The method of claim 8, wherein said immunohistochemistry analysis employs mAbP53 monoclonal antibody.

10. The method of claim 9, wherein said one or more markers is a protein or fragment thereof.

11. The method of claim 10, wherein said protein is an altered p53 protein or fragment thereof.

12. A method of detecting one or more markers indicative of cancer in an individual, comprising:
(a) obtaining a suitable biological sample;
(b) isolating (i) nucleic acid and (ii) tissue sample from said biological sample of (a);
(c) subjecting said isolated nucleic acid of (b) to methylation-specific PCR;
(d) subjecting said tissue sample of (b) to immunohistochemistry analysis; and
(d) detecting said one or more markers in said individual.

13. The method of claim 12, wherein said markers are selected from the group consisting of SFRP1, SFRP2, SFRP4, SFRP5, p53, and a combination thereof.

14. The method of claim 13, wherein said SFRP1, SFRP2, SFRP4, SFRP5 are methylated.

15. The method of claim 13, wherein said p53 is an altered protein or fragment thereof.

16. The method of claim 13, wherein said p53 is an altered protein or fragment thereof.

17. The method of claim 12, wherein said cancer is bladder cancer.

18. The method of claim 12, wherein said methylation-specific PCR is performed using primers selected from the group consisting of SEQ ID NOs. 1-16, and combinations thereof.

19. The method of claim 12, wherein said immunohistochemistry analysis employs mAbP53 monoclonal antibody.

20. The method of claim 12, wherein said biological sample is muscle from a bladder of said individual.
